(19) United States
(12) Patent Application Publication
(10) Pub. No.: US 2006/0246014 A1
(43) Pub. Date: Nov. 2, 2006

(54) Hairscents

(76) Inventor: Jaele Davis, Westchester, CA (US)

Correspondence Address: FULWIDER PATTON, 6060 CENTER DRIVE, 10TH FLOOR, LOS ANGELES, CA, 90045, US

(21) Appl. No.: 11/414,809

(22) Filed: May 1, 2006

Related U.S. Application Data
(60) Provisional application No. 60/676,982, filed on May 2, 2005.

(51) Int. Cl. A61K 8/64

(52) U.S. Cl. 424/47; 424/70.14

(57) ABSTRACT

A product for eliminating odors that can accumulate in a person's hair.

### CROSS-REFERENCES TO RELATED APPLICATIONS

This application is based on Provisional Application Ser. No. 60/676,982, filed May 2, 2005.

### BACKGROUND OF THE INVENTION

Environmental and personal factors can add undesirable odors to otherwise clean hair. Often, individuals go to a restaurant or other public place and when they leave, their hair smells like cigarette smoke, cooking oils and food, pollution or other environmental elements. Certain hairstyles, such as braids, braided or bonded weaves, beaded designs and dreadlocks, prohibit daily washing due to their intricate nature, but it is also great for hairstyles that require blow drying, flat ironing and wavy styles. It is great for people who work out in the gym regularly and want to freshen up their hair as they continue on with their errands and don't have time to wash their hair or go to the hair salon. Currently, no products are offered that provide a fresh scent and eliminate common odors that can occur between washings.

### SUMMARY OF THE INVENTION

HairScents is designed to eliminate odors that can accumulate on the hair in between washes. This unique invention contains ingredients that add protein and vitamins to the hair while eliminating the odors that become trapped in the hair follicles. This produce is offered in a variety of scents sure to please all consumers.

### BRIEF DESCRIPTION OF THE DRAWINGS

FIG. 1 is a schematic representation, depicting the present invention directed towards hair scents.

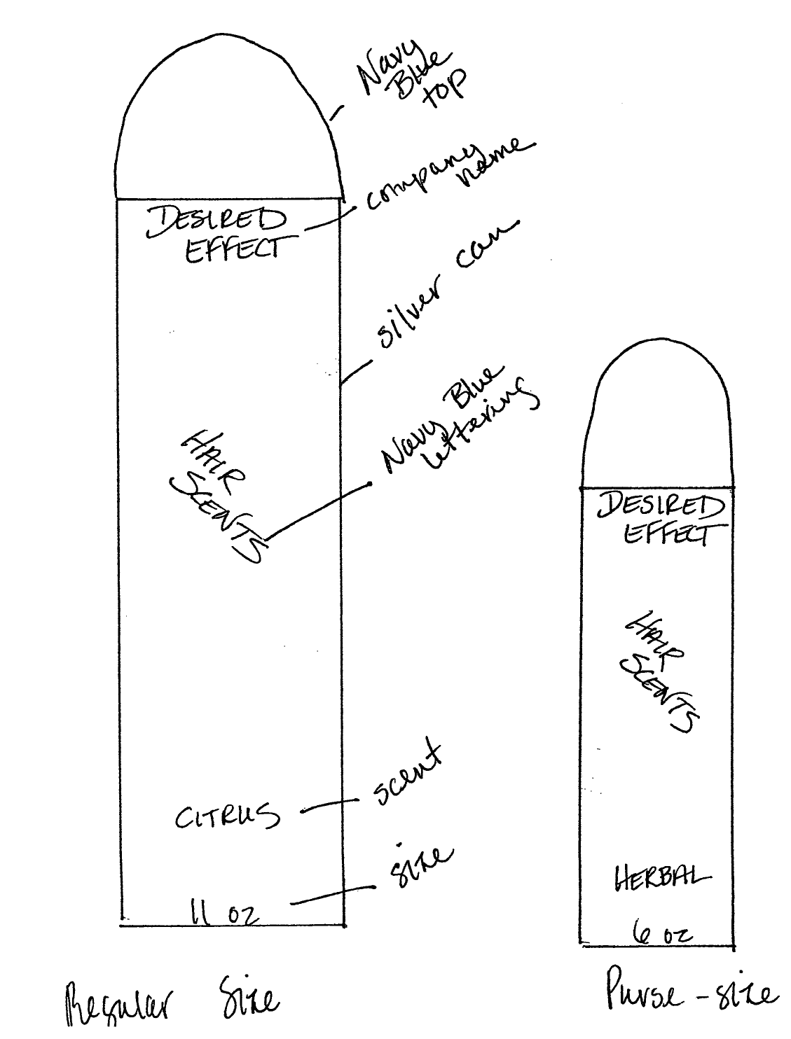

### DETAILED DESCRIPTION OF THE PREFERRED EMBODIMENTS

HairScents is designed to refresh the aroma of hair between washes. This product is a spray-on formula that can be offered in a variety of sizes. The typical sizes will include a container that is approximately 10″ tall an a smaller purse size can/bottle that is expected to be between 5-7″ in height. The spray contains ingredients that add protein and vitamins to the hair with no heavy product build-up. Scents are added to the formula to result in the hair smelling clean and fresh. The scents can include, but are not limited to: baby powder, citrus, fresh flowers and herbal fragrances. The container can be offered with a distinctive design that has an aluminum or silver appearance with navy blue and black lettering on the can. A blue cap or top may also distinguish this product from other hair care products on the market. To use: simply spray throughout dry hair and style as usual.

We claim:

1. A hair product, comprising:
a container, the container including means for spraying material stored in the container; and
a spray-on formula stored in the container, the formula including a protein and a plurality of vitamins wherein the formula eliminates odors trapped in hair follicles.
